(19) United States
(12) Patent Application Publication
(10) Pub. No.: US 2004/0267121 A1
(43) Pub. Date: Dec. 30, 2004

(54) Device and method for biopsy guidance using a tactile breast imager

(76) Inventors: Armen P. Sarvazyan, Lambertville, NJ (US); Vladimir Egorov, Princeton, NJ (US); Sergiy Kanilo, Lawrenceville, NJ (US)

Correspondence Address: Boris Leschinsky, P.O. Box 72, Waldwick, NJ, 07463, US

(21) Appl. No.: 10/866,396

(22) Filed: Jun. 12, 2004

Related U.S. Application Data
(60) Provisional application No. 60/477,741, filed on Jun. 12, 2003.

(51) Int. Cl. A61B 5/103; A61B 10/00

(52) U.S. Cl. 600/439; 600/587

(57) ABSTRACT

A biopsy guidance device is enclosed based on a tactile imaging probe adapted to accept a biopsy gun. The tactile imaging probe includes a pressure sensing surface providing real-time 2-D images of the underlying tissue structures allowing to detect a lesion. A cannula is provided supported at a center point by a ball and socket joint. The joint is equipped with linear and angular sensors and supports the cannula with the ability to rotate thereof about the center point. The position, linear and angular displacement and direction of the needle tip of a biopsy needle placed inside the cannula is therefore known at all times and provided as a feedback signal to a physician. Also provided to a physician is a position of the target site at a lesion, as well as a linear and angular deviation of the needle tip away from the target site. Such audio, light, or visual feedback allows the physician to correct the insertion angle and depth to confidently reach the target site to perform a biopsy. Method is also disclosed to guide the biopsy procedure.

### CROSS REFERENCE DATA

A priority date benefit is claimed herein from a U.S. Provisional Patent Application No. 60/477,741 filed on 12 Jun. 2003 by the same inventors and entitled “Biopsy guidance by tactile breast imager”, which is incorporated herein in its entirety by reference.

This invention was made with government support under SBIR Grants No. R43 CA91392 and No. R43/44 CA69175 awarded by the National Institutes of Health, National Cancer Institute. The government has certain rights in this invention.

### BACKGROUND OF THE INVENTION

1. Field of the Invention

This invention relates to methods and apparatus for performing image-guided biopsy of breast tissue. More specifically, the device and method of the invention relate to guidance using mechanical imaging obtained from tactile pressure sensor arrays.

2. Discussion of Background

Biopsy is the only definitive way to determine whether cancer is present in a suspicious area of tissue. A typical example of soft tissue is breast tissue. For the purposes of this specification, the term “breast” indicates any suitable soft tissue areas in need of diagnosis for a possible lesion. If a breast abnormality is detected with mammography or physical exam, a woman will typically be referred for additional breast imaging with diagnostic mammography, ultrasound, or other imaging tests. Depending on the results of these imaging tests, she may be referred for a breast biopsy. Known biopsy methods range from minimally invasive techniques, such as fine needle aspiration (using, for example, a 21 gauge hypodermic needle) and large core biopsy (using, for example, a 14 gauge needle mounted in an automated biopsy gun), to open-procedures in which the lesion is surgically excised. Minimally invasive techniques are faster, less expensive, safer and less traumatic for the patient than surgical excision, and have begun developing widespread acceptance. The introduction of image guided percutaneous breast biopsies offers alternatives to open surgical breast biopsy. Biopsy guns were introduced for use in conjunction with various guidance systems. Accurate placement of the biopsy gun is important to obtain useful biopsy information because only one small core could be obtained per insertion at any one location. To sample the tissue thoroughly, many separate insertions of the instrument are often required.

A critical issue in conducting of core biopsies is the accurate location of a lesion and thereafter the accurate guiding of the biopsy needle to that lesion. Sophisticated methods and apparatus have been developed for core biopsies in connection with mammography/ultrasound. Stereotactic breast biopsy involves taking of a first radiographic image of a lesion in a breast, moving the breast or x-ray tube a known distance and then taking a second radiographic image of the lesion in the breast, so that the x, y and z coordinates of the lesion site in the breast may be calculated. Once the location of the lesion has been confirmed, a biopsy needle and associated biopsy gun are placed with computer assistance to place the biopsy needle to the calculated position in the breast. The use of such equipment is expensive, time consuming, and cannot be justified in small practices.

A common drawback of all of the mammographic biopsy guidance systems is the need for multiple X-rays of the tissue, thus exposing the tissue to potentially unhealthy ionizing radiation. These systems also provide no real-time imaging of the needle trajectory as it enters the breast. Intervening movement of the breast tissue may render the calculated coordinates useless and result in a potentially misleading biopsy sample. Indeed, the clinician is not even aware that the biopsy needle missed the intended target until the follow-up stereotactic views are taken.

Moreover, because the biopsy needle is secured in a fixed housing so as to provide a fixed trajectory for biopsy needle, stereotactic systems provide no freedom of movement for the biopsy needle relative to the target tissue. Consequently, several needle insertions and withdrawals are required to adequately characterize the tissue.

Mammography needle biopsy devices are shown and disclosed in U.S. Pat. Nos. 5,526,822, 5,649,547, 5,769,086, and 6,280,398. Commonly used needle biopsy device, known commercially as the MAMMOTOME Biopsy System, which is available from Ethicon Endo-Surgery, Inc., a division of Johnson & Johnson, has the capability to actively capture tissue prior to cutting the tissue sample. Active capture allows for sampling through non-homogeneous tissues. The device is comprised of a disposable probe, a motorized drive unit, and an integrated vacuum source. The probe is made of stainless steel and molded plastic and is designed for collection of multiple tissue samples with a single insertion of the probe into the breast. The tip of the probe is configured with a laterally disposed sampling notch for capturing tissue samples. The device employs a computer-digitizer system to digitize the location of a point of interest within the patient's breast as that point of interest appears on a pair of stereo x-rays of the breast. Thereafter, the device computes the three-dimensional or spatial coordinates of that point of interest and displays them to the user. Orientation of the sample notch is directed by the physician, who uses a thumb-wheel to direct tissue sampling in any direction about the circumference of the probe. A hollow cylindrical cutter severs and transports tissue samples to a tissue collection chamber for later testing.

Another example of a system and process for performing a percutaneous biopsy within the breast using three-dimensional ultrasonography is described in the U.S. Pat. No. 6,254,538. This system uses three-dimension ultrasonography and includes a breast positioning device, a breast immobilization device, an ultrasonic imaging device, and a biopsy instrument positioning device, all adapted to assist an operator in guiding a biopsy instrument percutaneously to the lesion.

Another yet ultrasound-guided biopsy apparatus and methods are disclosed in U.S. Pat. No. 5,833,627. Positioning of a needle or cannula of a biopsy device for insertion into a tissue mass is achieved by correlating, in real-time, the actual needle or cannula position prior to insertion with its probable trajectory once inserted. In a preferred embodiment, a biopsy device support block is mechanically coupled to an ultrasound transducer to provide alignment of the biopsy device with the ultrasound image in at least one plane. Continued ultrasound scanning of a selected trajectory may be provided to assess the depth of penetration of the needle or cannula of the biopsy device, when inserted.

An optical-guided biopsy system and corresponding methods are described in the U.S. Pat. No. 6,174,291. A system characterizes tissue using fluorescence spectroscopy, such as light-induced fluorescence. Native fluorescence from endogenous tissue without requiring fluorescence-enhancing agents is used to distinguish between normal tissue, hyperplastic tissue, adenomatous tissue, and adenocarcinomas. The system provides endoscopic image enhancement for location of a tissue site for optical biopsy tissue characterization and biopsy guidance. The system allows the use of an integrated endoscopic diagnosis and treatment device for immediate diagnosis and treatment without interchanging equipment and relocating the tissue site. The system is also integrated with existing endoscopy equipment for initiating and displaying the diagnosis. The system provides an adjunctive tool to histopathological tissue classification or, alternatively, further treatment is based on the optical biopsy system diagnosis itself.

Magnetic resonance imaging (MRI) is widely used for detection of breast malignancies that have previously been sub-clinical (i.e., neither palpable nor detected by mammography). Stereotactic MRI-guided breast biopsy method and device are described in the U.S. Pat. No. 5,706,812. In this device, an MRI breast coil is provided with a large transverse access portal and a stereotactic frame for guiding a biopsy needle. First coil portion is located about the distal portion and the second coil portion is located about the proximal portion thereof. The portal is covered by a thin sheath of plastic to retain the breast but still allow insertion of the needle into any location. The frame aligns the needle by azimuth, height, and depth.

An image-guided breast lesion localization device and biopsy system is described in the U.S. Pat. No. 5,855,554. It employs a chest support for holding the patient in a slightly rotated prone position allowing the breast tissue to hang downward and fit through an opening in the chest support, while holding the other breast against the subject away from the imaging region. The chest support is retrofitted to existing tables of medical imaging devices such as magnetic resonance, X-ray, ultrasound or computer tomography imaging devices. A pair of support plates is used to compress the breast tissue. At least one of the support plates has a grid with reference markers for localization as well as windows allowing a physician access to the breast tissue. A thick biopsy plate with a plurality of holes at marked positions fits into one of the grid openings and guides an interventional device, such a biopsy needle, into a desired location in a lesion.

Despite the incredible available imaging power of existing technologies, very few procedures are actually done using imaging devices in a routine clinical setting. There are several reasons for the lack of general acceptance of these devices in existing markets. Most of the systems are expensive, and normally this expense cannot be justified in terms of usage or benefit for the capital investment required.

Palpation method of performing a biopsy on a suspicious structure in tissue including preliminary palpation by a clinician to first locate the structure is widely used. Upon locating the structure, the clinician uses her fingers to constrain the structure. Once the clinician has done so, the biopsy needle is inserted into the tissue to the approximate depth of the structure. As the needle penetrates the outside portion of the mass, the clinician senses a slight increase in resistance against the needle, which confirms that the needle has reached the structure. Because the clinician does not know the form and depth of the structure for certain, obtaining a good sample of the tissue or the fluid inside the structure typically involves some trial and error. The clinician may insert and reinsert the needle multiple times to ensure that a good sample has been obtained.

U.S. Pat. Nos. 5,833,633 and 6,468,231 (incorporated herein by reference in their entirety) describes the use of MI for biopsy guidance where an embodiment is made up of an electronically controlled mechanical scanning unit incorporated into a patient support bed. The mechanical scanning unit includes a compression mechanism and positioning system, a local pressure source located opposite a pressure sensor array, and electronic control and interface circuitry. The local pressure source is either a roller moving over the examined breast, or in another embodiment, a so-called “indenter”, which can be moved in all three dimensions and be controlled either automatically by a computer or manually with a mouse. In another embodiment, the mechanical scanning system serves as biopsy guidance means and determines target lesions in the breast to be reached by a biopsy gun or aspiration needle.

U.S. Pat. No. 6,468,231 also incorporated herein by reference describes various hand-held tactile imaging probes equipped with a pressure sensor array. No provisions are mentioned to use these probes for biopsy guidance.

There has been another attempt (U.S. Pat. No. 6,063,031) to develop a palpation device for sensing regions of hardening in the breast tissue and using the palpation data for tissue biopsy. The device is provided for diagnosis and treatment of tissue with instruments, specifically locating a tissue structure and positioning an instrument relative to that tissue structure. The device includes a plurality of sensors for generating signals in response to pressure imposed on the sensors as the sensors are pressed against the tissue. The device also includes a member configured to be pressed against tissue and containing sensors for detection of an underlying tissue structure in the tissue. The device also includes a locating device, arranged at a selected position with respect to the sensors, for indicating a location of the underlying tissue structure. The method of using the device includes positioning the locating device, which may be an instrument guide, over an underlying tissue structure based on the image. The method further includes using the locating device to direct the insertion of an instrument for treating or diagnosing that tissue structure. The embodiments of the method of this patent are complex and physically large and may have a limited application.

New methods and devices are therefore needed which avoid the high cost equipment, and which may be conducted relatively quickly and efficiently, whilst maintaining accuracy of the biopsy.

### SUMMARY OF THE INVENTION

It is therefore an object of the present invention to overcome the disadvantages of the prior art and provide a method and apparatus for core biopsies.

It is another object of the invention to is to provide a simple method and an inexpensive device

for imaging the breast, detecting lesions, and guiding biopsy.

Another object of the invention is to provide biopsy guidance devices and methods utilizing physical principles and measured parameters similar to those associated with a physical examination by manual palpation conducted by a skilled physician.

A further yet objective of the invention is to provide biopsy guidance methods and devices allowing objective directing of a biopsy device towards the target location without relying on the subjective sensations of the operator.

Another yet objective of the present invention is to provide a biopsy guidance device with increased sensitivity, repeatability, and accuracy.

Biopsy guidance system of the present invention is based on a technology named “Mechanical Imaging” (MI) (see for example Sarvazyan A. P., Mechanical Imaging: A new technology for medical diagnostics.—Int. J. Med. Inf., 1998, 49, 195-216). It uses the same mechanical information as obtained by manual palpation conducted by a skilled physician. MI method and device provide detection of tissue heterogeneity and hard inclusions by measuring changes in the surface stress pattern using a pressure sensor array pressed against the tissue.

The above and other objects are achieved according to the present invention by providing a method for the detection of lesions in the breast tissue, including generation of a 3-D digital image from a sequence of 2-D tactile images; and extracting features that characterize a lesion in order to provide real time biopsy guidance.

This invention is based on a principle of guidance a biopsy needle by a tactile imager probe to a calculated site of an underlying target lesion. The device comprises a tactile sensor array having a plurality of pressure sensors, each of said sensors producing a signal in response to pressure imposed on the sensor as the sensors are pressed and moved against the breast tissue in a predetermined manner. The device further includes a spring-loaded biopsy gun controllably connected with said tactile sensor array by means of a cannula for insertion of a biopsy needle.

The needle is directed to an identified point of interest within the patient's breast for obtaining a specimen at a target biopsy site. The device further includes means for calculating reciprocal disposition between the target lesion and the site at which the needle is aiming, as well as a feedback signal means characterizing distance and relative orientation between the lesion and the target site.

A biopsy needle employed in a tactile imaging biopsy system of the invention is guided in accordance with coordinate information calculated in real-time. That information represents both an identified point of interest within a patient's breast and the biopsy needle position relative to a pressure sensing surface of a tactile breast probe. The probe is manually adjusted in accordance with that coordinate information to permit insertion of the biopsy needle to the identified point of interest.

### BRIEF DESCRIPTION OF THE DRAWINGS

FIG. 1 is a perspective view showing the use of a first embodiment of the breast biopsy guidance device with an audio feedback signal.

FIG. 2 is a perspective view showing the use of a second embodiment of the breast biopsy guidance device of the present invention with a liquid crystal display.

FIG. 3 is a perspective view showing the use of a third embodiment of the breast biopsy guidance device with light feedback signal.

FIGS. 4A and 4B are perspective views of a fourth embodiment of breast biopsy guidance device of the present invention.

FIG. 5A is a cross-sectional view of a sixth embodiment of the breast biopsy guidance device.

FIGS. 5B and 5C are schematic illustrations of the use of the sixth embodiment of the breast biopsy guidance device shown in FIG. 5A.

FIG. 5D illustrates a disposable sterile membrane provision covering the sensor array.

FIGS. 6A, 6B and 6C are schematic illustrations of manually controlled adjustment of the needle position in a biopsy guidance device with the ball and socket joint.

FIG. 7 is a flow chart of a method of biopsy guidance by the tactile imager.

FIG. 8 is a diagram illustrating the concept and functioning of the biopsy guidance by computerized tactile imager.

FIGS. 9A, 9B and 9C illustrate the principle of needle biopsy guidance using 2-D tactile images.

FIGS. 10A, 10B and 10C illustrate the principle of needle biopsy guidance using two orthogonal 2-D tactile images.

FIGS. 11A, 11B and 11C illustrate the principle of needle biopsy guidance using 3-D reconstructed tactile images.

FIG. 12 illustrates the concept of a self-directing biopsy needle tip.

### DESCRIPTION OF THE PREFERRED EMBODIMENTS OF THE INVENTION

Reference will now be made in greater detail to preferred embodiments of the present invention, examples of which are illustrated in the accompanying drawings. Wherever possible, the same reference numerals will be used throughout the drawings and the description to refer to the same or like parts.

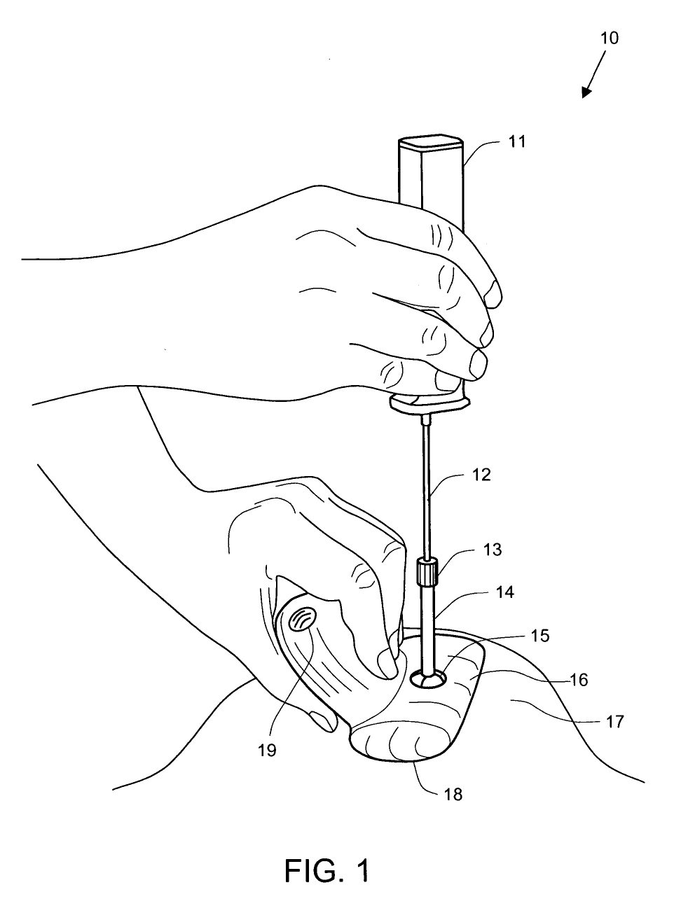

FIG. 1 shows a first embodiment of a hand-held breast biopsy device 10 during breast biopsy guidance in accordance with the present invention. Device 10 comprises a tactile sensor array or another appropriate pressure sensing means with a pressure sensing surface 18 having a plurality of pressure sensors. Each of the sensors produce a signal in response to pressure imposed on the sensor as the sensors are pressed and moved over the breast tissue in a predetermined manner. A spring-loaded or another type of a biopsy gun 11 is controllably connected with the tactile sensor array by means of a guiding cannula 14. The guiding cannula is positioned at a center point 15 located in a known relationship to the pressure sensing surface of the tactile imaging probe and can be rotated about this center point. The guiding cannula 14 accepts a biopsy needle 12 inserted therein. The term “biopsy gun” broadly means any suitable device to handle a biopsy needle including an aspirating syringe, a spring-loaded biopsy gun, a gas-driven biopsy gun, etc.

Computing means are provided to calculate reciprocal disposition between a detected lesion and the target site at which the needle is aiming. The term “reciprocal disposition” means geometrical information about the position of the target site and the position of the end of the needle. In a most direct case, this includes a deviation angle between the direction of the needle and the direction from the center point of the tactile imaging probe towards the center of the target site. The ball and socket joint 15 is considered to be a center point of the tactile imaging probe, since the biopsy gun and the needle can be rotated or rocked about that point as discussed in more detail below. In addition to the angle information, the term “reciprocal disposition” included a distance information between the end of the needle and the target site.

A feedback signal is also provided to indicate to the user the relative deviation angle and distance between the lesion and the target site at which the needle is aiming.

The general use and operation of the device 10 will now be described. A biopsy needle 12 is inserted into a cannula 14 optionally equipped with a tightening ring 13. The ball and socket joint located at the center point 15 allows adjusting the orientation of the needle relative to the sensing surface 18 of the tactile breast probe 16. The details of the ball and socket joint are described in more detail below. The operator can vary the depth of needle insertion in the cannula 14. The operator holds the biopsy gun 11 with one hand and the tactile imager probe 16 with the other hand. She places the tactile imager probe 16 with a pressure sensing surface 18 on a breast 17 above approximate location of the lesion, which was detected earlier by a known diagnostic modality (e.g. mammography, ultrasonography, magnetic resonance imaging, mechanical imaging). Preliminary identifying a suspicious region in the breast can also be performed using the tactile imager probe 16 itself without the biopsy gun 11.

During the local scanning over the suspicious region of the breast, the speaker 19 mounted in the handle of the tactile breast probe 16 produces a feedback sound signal indicating detection of the lesion and characterizing the distance between the lesion and the target site. There are numerous ways to guide the biopsy needle by the audio feedback signal. In one embodiment, the tone (the frequency) of the sound indicates the angle between the direction of the needle and the location of the lesion while the loudness of the sound indicates the distance between the lesion and the site at which the needle is aiming. The tone is the highest when the needle points towards the lesion and sound is the loudest when the needle aims at the center of the lesion. After the operator successfully completes the aiming the needle at the lesion, she presses the trigger of the biopsy gun and gets the sample.

Other embodiments of the biopsy guidance using audio feedback can be based upon other combinations of the parameters of the sound signal, which can be not only continuous but also pulsating or amplitude-modulated. In addition to the loudness and the tone, such parameters as the pulse repetition rate and spectral content can be used.

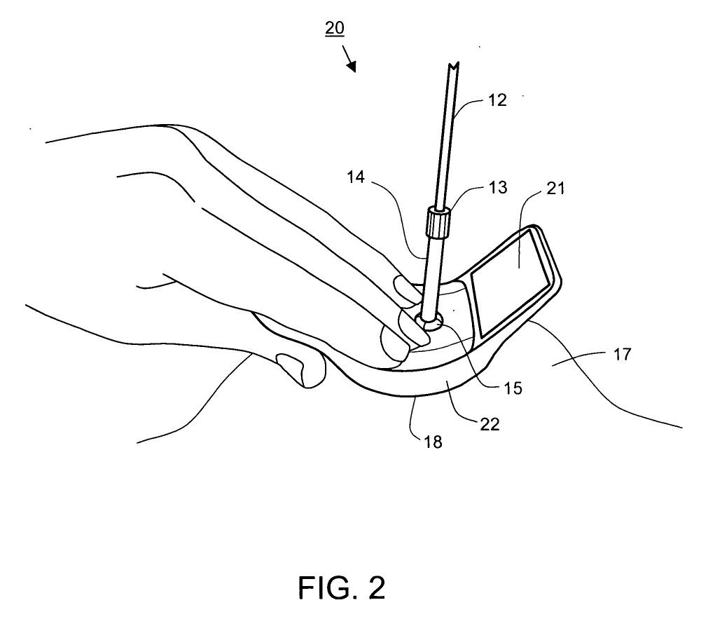

FIG. 2 illustrates a hand-held breast biopsy guidance device 20 in the accordance with a second embodiment of the present invention. The device 20 comprises the following elements:

a tactile sensor array with pressure sensing surface 18 having a plurality of pressure sensors, each of said sensors producing a signal in response to pressure imposed on the sensor as the sensors are pressed and moved over the breast tissue in a predetermined manner,

a spring loaded biopsy gun controllably connected with the tactile sensor array by means of a cannula 14 with a tightening ring 13 for insertion of a biopsy needle 12,

a computing means for calculating reciprocal disposition between the detected lesion and the target site at which the needle is aiming, and

a liquid crystal display 21 for biopsy guidance in real-time by visualizing the relative orientation and the distance between the lesion and the target site.

In use, the operator places the tactile imager probe 22 on the breast 17 above approximate location of the lesion and scans the tactile imager probe to visualize the lesion on the display 21. Then the operator advances the biopsy gun while adjusting the position of the biopsy needle 12 inserted into a cannula 14 by observing a mutual position of the lesion and tissue site.

The ball and socket joint 15 allows adjusting the angular orientation of the needle relative to the sensing surface 18 of the tactile breast probe 22. The operator can also vary the depth of needle insertion in cannula 14. When the operator matched the image of the lesion on the display with needle markers, she releases the spring of the biopsy gun to get a biopsy sample.

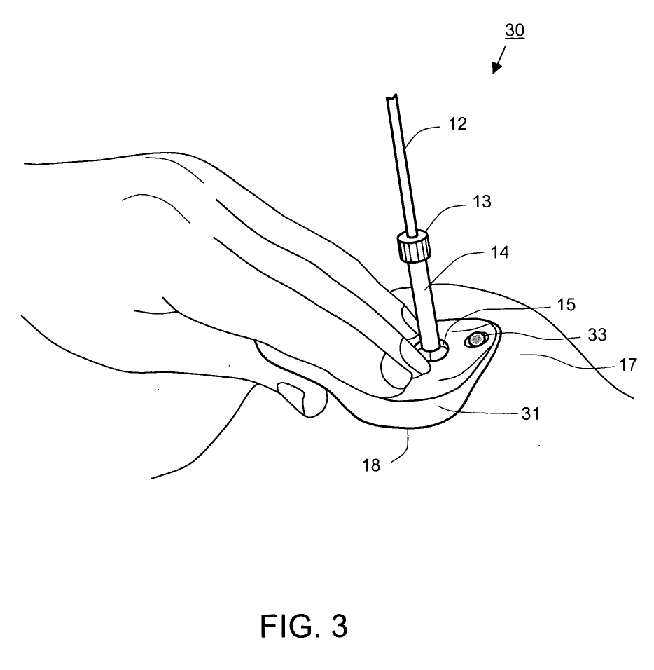

FIG. 3 shows a hand-held breast biopsy guidance device 30 in the accordance with a third embodiment of the present invention. Device 30 includes:

a tactile sensor array with pressure sensing surface 18,

a spring-loaded or another type of a biopsy gun controllably connected with the tactile sensor array by means of a cannula 14 for insertion of a biopsy needle 12,

a computing means for calculating reciprocal disposition between a detected lesion and the target biopsy site, and

a light indicator 33 for communicating the angle and distance information to the operator.

In use, the operator places the tactile imager probe 31 on the breast 17 above approximate location of the lesion. Then operator aims the needle 12 of a biopsy gun at the center of the lesion in accordance with feedback light signal emitted from the light indicator such as an LED 33. There are several conceived possibilities to guide the biopsy needle by the feedback light signal. The information on the relative location of the lesion with regard to the region at which the needle is aiming can be coded by such parameters of the light indicator as its brightness, color and pulse repetition rate (PRR). For example, the PRR may indicate the angle between the direction of the needle and the location of the lesion and the brightness of the light may indicate the distance between the lesion and the site at which the needle is aiming. The PRR is the highest when the needle is pointing towards the lesion and light is the brightest when the needle is aiming at the center of the lesion.

After the operator successfully completes the aiming the needle at the lesion she activates the biopsy gun by pressing on the trigger and gets the tissue sample.

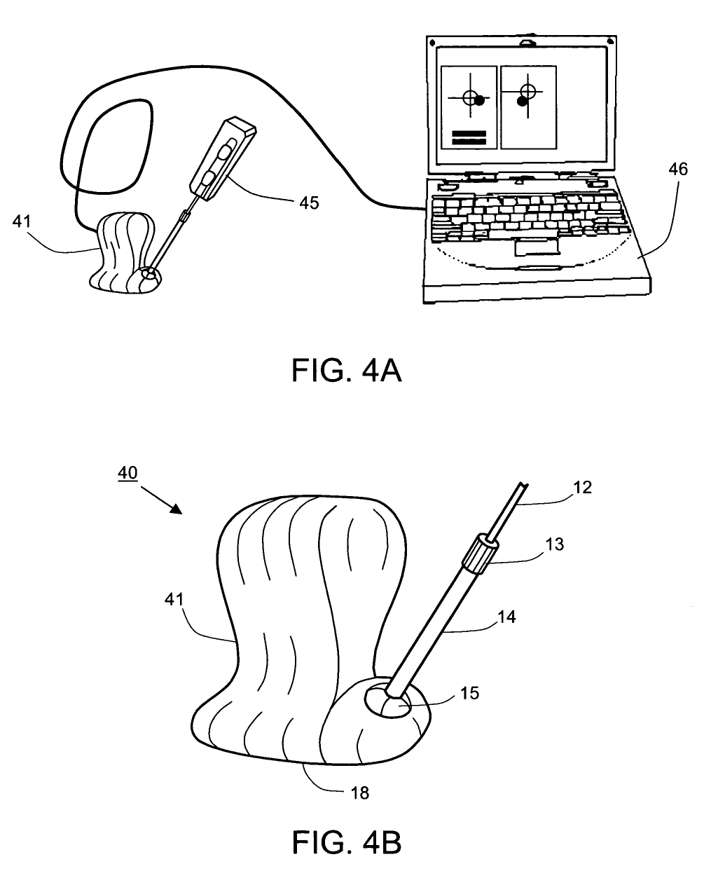

FIGS. 4A and 4B show a general view of computerized breast biopsy guidance device 40 in the accordance with the fourth embodiment of the present invention. The device 40 comprises a tactile imager probe 41 with biopsy gun 45 and computer 46. The tactile imager probe 41 further comprises a tactile sensor array with pressure sensing surface 18, and a cannula 14 for insertion of a biopsy needle 12 to obtain a tissue specimen at a target biopsy site. Reciprocal disposition between a detected lesion and the target biopsy site are calculated in real-time by means of computer 46 and accordingly displayed on a computer screen to guide the aiming of the needle.

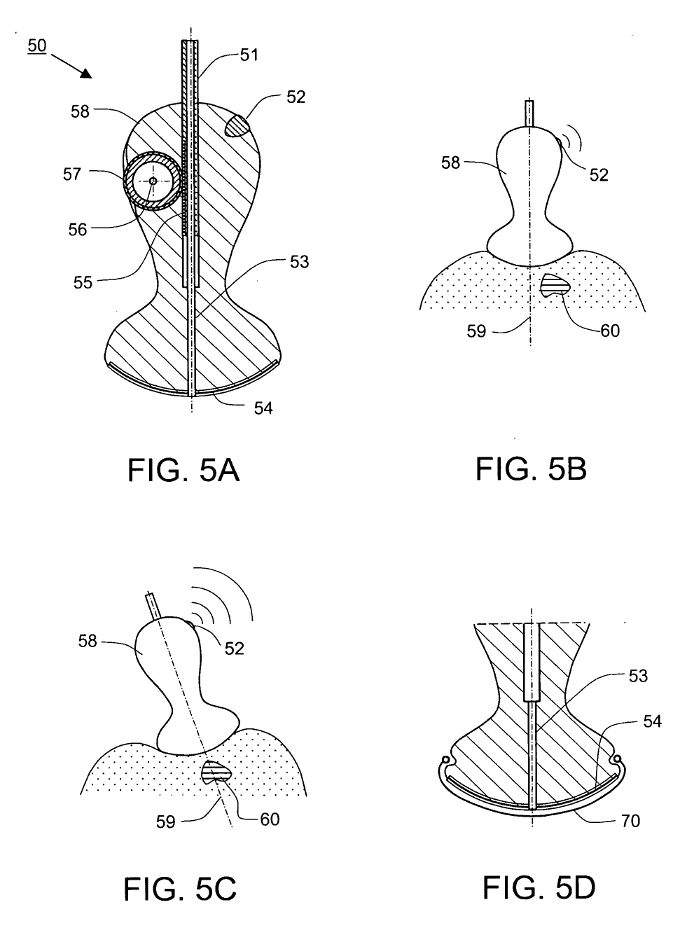

FIG. 5A shows a cross-section of a hand-held breast biopsy guidance device 50 in accordance with a fifth embodiment of the present invention. Device 50 includes the following elements:

a tactile sensor array with a preferably round concave pressure sensing surface 54 having a plurality of pressure sensors,

a vertical movable cannula 51 for insertion of a biopsy needle,

a control thumb-wheel 57 mounted inside the housing 58,

an opening channel 53 for passing through the biopsy needle, and

a feedback signal means 52, which can be either a light source or a sound source, similar to the feedback signal means described in the embodiments shown in FIGS. 1 and 3.

The needle inserted into the cannula stays in the fixed position relative to the cannula. The wheel 57 has a gear ring connected to a toothed part 55 of the cannula 51 so that the movable cannula 51 and the wheel 57 form a gear pair for transforming a wheel rotation into forward movement of the cannula 51. As can be well appreciated by those skilled in the art, other mechanical advancement means can be used in place of the gear mechanism such as a sliding mechanism, a worm-gear rotating mechanism etc. Importantly, such mechanical advancement means must have a linear displacement sensor for measuring the current position and the distance of advancement of the needle and continuously feeding that information into the computing means of the device.

Referring to FIGS. 5B and 5C, the operation of the device 50 will now be described. An operator places (preferably with one hand) the biopsy guidance device 50 over the approximate location of the lesion as detected earlier by any known diagnostic modality (e.g. mammography, ultrasonography, magnetic resonance imaging, mechanical imaging). Such prior identifying a suspicious region in the breast can also be performed using the device 50 without biopsy gun. During the local scanning over the suspicious region of the breast, the speaker 19 (or alternatively, a light diode or another indicator) mounted in the tactile breast probe 16 produces a feedback signal (such as tactile, audio or light) indicating lesion detection. The indicator is also used to communicate the angle and distance information between the lesion and the site at which the needle is aiming. In the case of audio feedback signal, the deviation angle between the direction of the needle and the direction towards the target site can be coded by, for example, the tone of the signal. At the same time, the linear distance between the center of the target and the point where the needle is aiming at can be coded either the amplitude of the sound or by the pulse repetition rate of that sound. A combination of indicators is also envisioned for this and other embodiments of the invention.

Guided by a feedback signal 52, the operator adjusts the angular position of the probe 58 to direct its axis 59 towards the target lesion 60, as shown in FIGS. 5B and 5C. After the axis 59 is adjusted and the needle is directed towards the lesion, the feedback signal is used to guide the adjustment of the penetration depth of the needle by turning the thumb-wheel 57. After the guidance process is accomplished, the operator presses the trigger of the biopsy gun and gets the sample from the lesion 60. The embodiment shown in FIGS. 5A, 5B, and 5C can be optionally made as an attachment to a biopsy needle gun.

FIG. 5D shows an additional feature of the tactile imager, such as the one illustrated in FIGS. 5A, 5B, and 5C—a disposable, sterile thin elastic membrane 70 covering the sensor surface 54 contacting the patient's breast.

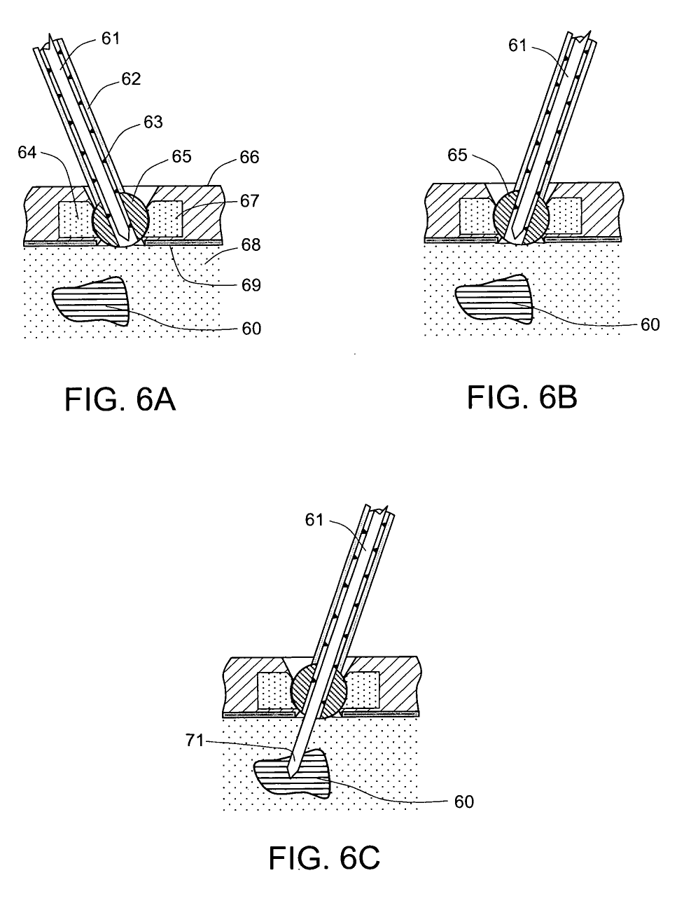

The embodiments of the biopsy guidance device shown in FIGS. 1, 2, 3, 4A, and 4B all have one similar element: a ball and socket joint 15 that allows for adjustment of the position of the needle relative to the sensor array. FIG. 6A shows a cross-sectional view of a cannula 62 with a ball and socket joint 65. Linear displacement sensor 63 and angular displacement sensors 64, 67 are all mounted inside the cannula 62 and the corresponding housing 66 in the vicinity of the tactile sensor array 69. FIGS. 6A, 6B, and 6C show successive steps of the needle 61 aiming at the target lesion 60 in the breast tissue 68.

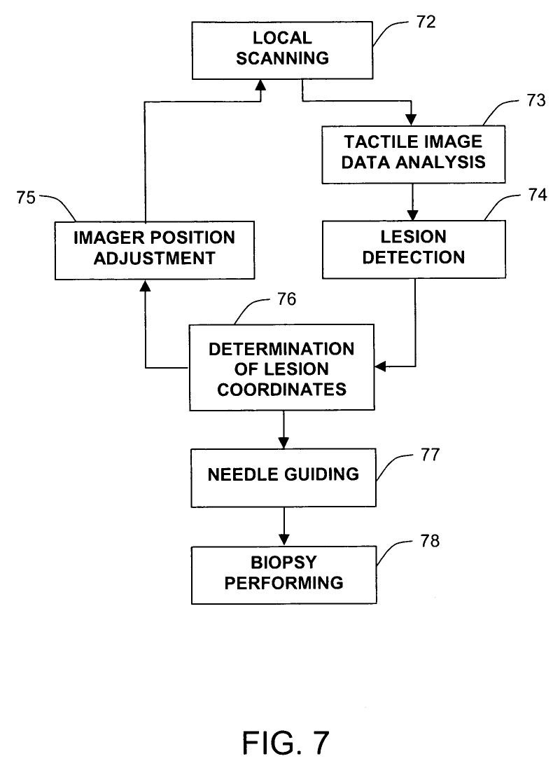

FIG. 7 shows a flow chart illustrating the method of biopsy guidance according to the present invention by using a tactile imager with a biopsy gun attached. The first step is a local scanning 72 over approximate location of the lesion. Specialized software of the computing means of the device analyzes the acquired sequence of 2-D tactile images 73, detects the lesion 74 and provides an audio or light signal indicating the presence of the lesion and its location relative to the tactile imager. If the lesion is far from an optimal position relative to the tactile imager and the biopsy gun, the position of the tactile imager is adjusted in step 75 accordingly and the loop of steps 72, 73, 74 and 75 is repeated. In the step 76, the lesion coordinates are calculated and the feedback signal is produced enabling the operator to guide the needle to the target area 77. After the feedback signal indicates that the needle is aiming at the center of the lesion, the trigger of the biopsy gun is pressed and a biopsy sample is collected in the step 78.

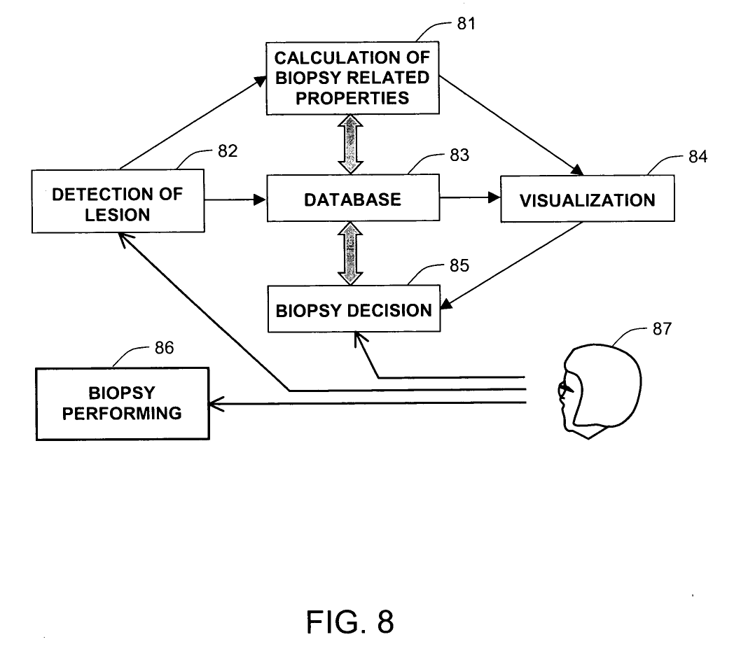

FIG. 8 illustrates the second method of the present invention advantageously implemented in a computing means of the embodiment illustrated in FIG. 4A. In that case, the tactile imaging probe data is used to visualize the lesion, calculate mechanical and geometrical features of the lesion, relate these features to the database of breast tissue biomechanics and breast pathology, and display the results of such a computerized analysis. The physician will be able to use the system in a dialog mode to test her own diagnostic hypotheses before biopsy.

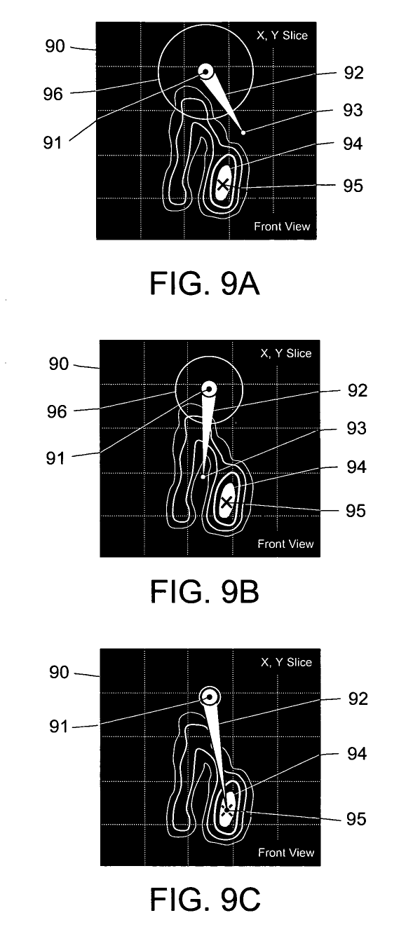

FIGS. 9A, 9B and 9C illustrate the general principle of biopsy needle guidance by means of 2-dimensional projection of underlying breast tissue. Referring to FIG. 9A, a display 90 shows a 2-D projections of the point 91 on the breast surface at which the needle is placed before insertion. Also displayed are the direction of the needle, the point inside the breast at which the needle is aiming at a particular moment in time 93, the lesion 94 and the center of the lesion at which the biopsy sample should be collected 95. The diameter of the circle 96 indicates the linear distance between the points 95 and 93, that is the distance between the target and the point at which the needle is aiming. The closer the position of the needle is to the final position required for optimal tissue sampling, the smaller is the diameter of the circle 96. The needle navigation is being accomplished by continuous real-time monitoring of the spatial relationship between the biopsy needle and the breast lesion under the guidance provided by the display 90. An operator must superimpose the tip of a virtual needle 93 with the lesion center 95 and achieve minimum diameter of the circle 96. When the needle is aimed directly at the target 95, the diameter of the circle 96 becomes close to zero. FIG. 9A shows that the virtual needle tip is far from the lesion center. On the FIG. 9B the virtual needle is brought closer, and FIG. 9C shows that final position when the virtual needle is aiming straight at the lesion center. That means that the biopsy gun may be activated.

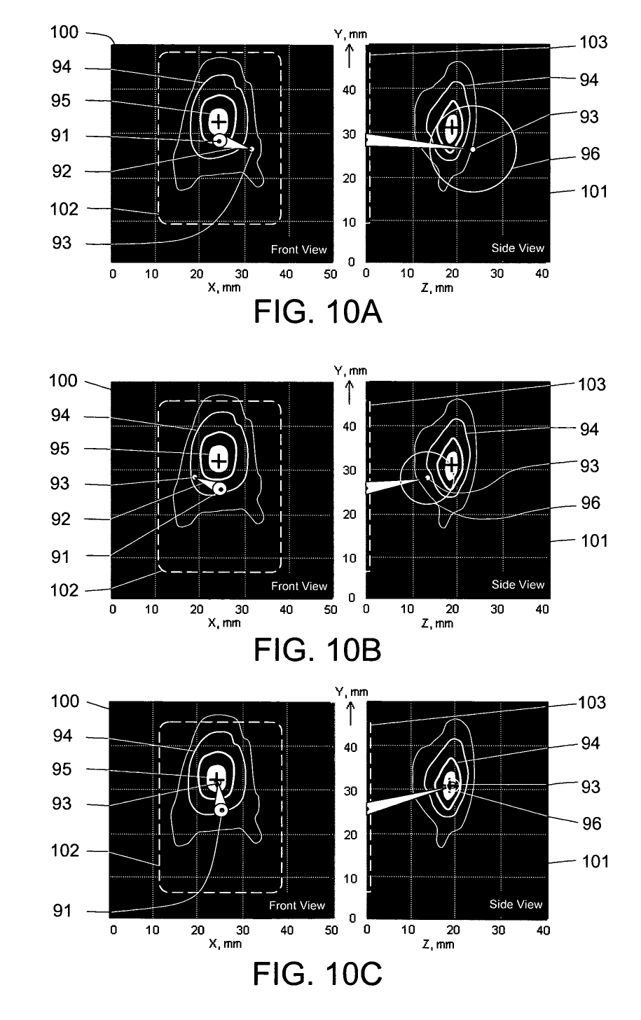

FIGS. 10A, 10B and 10C illustrate another general method of biopsy needle guidance by means of two orthogonal projections of underlying breast tissue. Referring to FIG. 10A, a horizontal projection (parallel to skin surface/front view) 100 and vertical projection (perpendicular to skin surface/back view) 101 of 3-D tactile image of underlying tissue are represented. The front view screen 100 displays pressure sensing area contour 102 of the tactile breast imager, a virtual needle direction 92, and a projection of the lesion 94 with a center 95. The side view screen 101 displays pressure sensing area base of the tactile breast imager 103, a virtual needle aiming at the point 93, and a projection of the lesion 94. The diameter of the circle 96 with the center in the virtual needle tip 93 corresponds to the linear distance from a target biopsy site to the point 93 at which the needle is aiming. The closer the virtual needle tip is to the optimum biopsy position, the smaller is the diameter of the circle 96. Needle navigation is accomplished by real-time monitoring of the spatial relationship between the biopsy needle and the breast lesion under guidance provided by the projections 100 and 101. An operator must superimpose the tip of virtual needle 93 with the lesion center 95 and achieve minimum diameter of the circle 96. When the needle is aimed directly at the target 95, the diameter of the circle 96 becomes close to zero. FIGS. 10A, 10B and 10C illustrate the process of biopsy guidance. FIG. 10C shows the final position when the virtual needle is aiming straight at the lesion center. At that position the operator presses the trigger of the biopsy gun and collects a tissue sample.

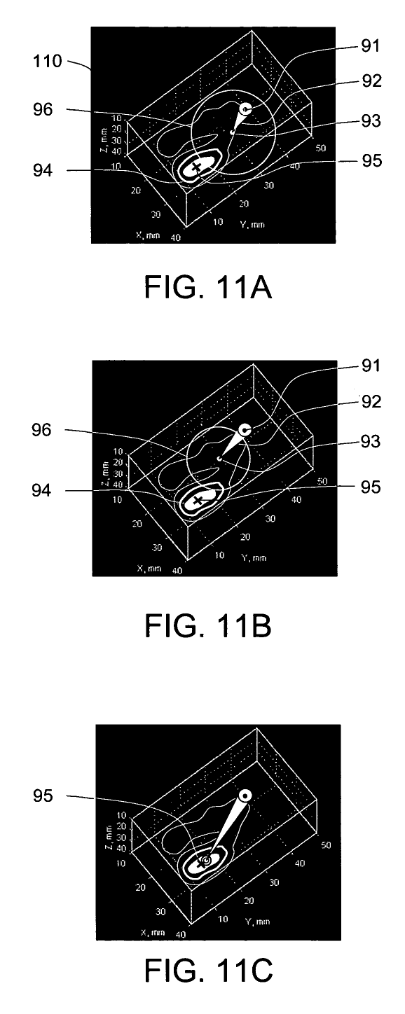

FIGS. 11A, 11B and 11C illustrate yet another method of biopsy needle guidance by means of three-dimensional tactile image of underlying breast tissue. FIG. 11A illustrates a display 110 showing a virtual needle origin 91, the needle direction 92, the point inside the breast at which the needle is aiming an a particular moment in time 93, the lesion 94 and the center 95 of the lesion, at which the biopsy sample should be collected. The diameter of the circle 96 indicates the linear distance between the points 95 and 93, that is the distance between the target and the point at which the needle is aiming. The closer the position of the needle is to the final biopsy position, the smaller is the diameter of the circle 96. The needle navigation is accomplished by continuous real-time monitoring of the spatial relationship between the biopsy needle and the breast lesion under the guidance provided by display 90. An operator must superimpose the tip of virtual needle 93 with the lesion center 95 to achieve minimum diameter of the circle 96. When the needle is aimed directly at the target 95, the diameter of the circle 96 becomes close to zero. FIG. 11C shows that final position when the virtual needle is aiming straight at the lesion center and the system is ready for collecting a biopsy sample from the target area.

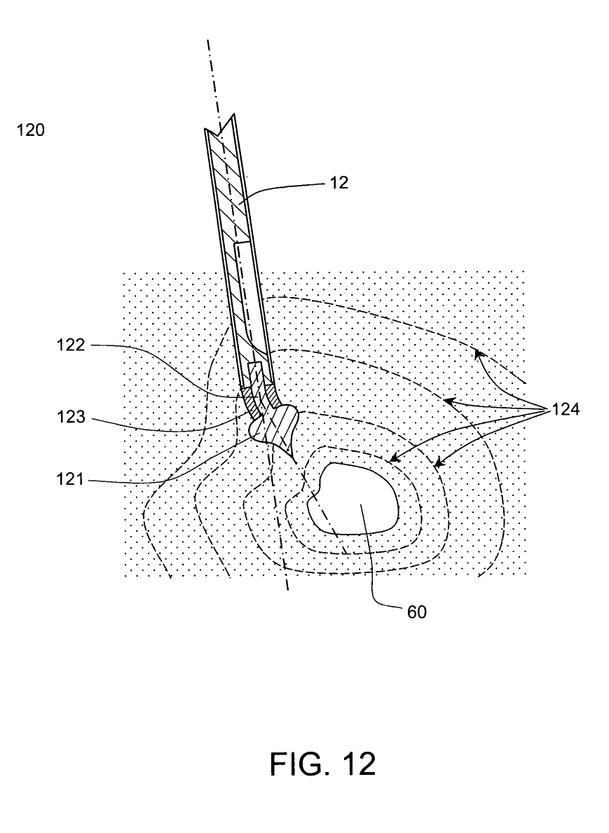

The technology of biopsy guidance using a tactile imager is based on the fact that the majority of breast cancers are palpable, that is that the target lesion is harder than the normal surrounding tissue. Tactile imaging device provides a possibility of detecting hard lesions and evaluating their coordinates. Inevitably, the evaluation of the position of the lesion relative to the tactile imager probe is made with a certain error. To further increase the success of the biopsy procedure, it is important to minimize the error in evaluating the exact position of a hard lesion. An alternative is proposed here to provide an additional guidance means to the system of the biopsy needle and the tactile imager, which will increase a possibility of directing the needle exactly to the region of elevated hardness. Such additional guidance means enhancing the ability of the system to direct the needle closer to the center of the hardest area in the breast is illustrated in FIG. 12. Position error in calculating at the exact location of the lesion and can be corrected by a self-directing extension of the needle.

More specifically, FIG. 12 shows a detachable and disposable guiding extension 121 to the biopsy needle 12 that provides a possibility of self-directing of the needle towards the region of the greatest hardness. The guiding extension 121 serves as a means to increase the probability of getting the biopsy sample exactly from the region with the highest elasticity modulus. Lines 124 schematically show the gradient of the tissue hardness. It is assumed that in a certain limited area in the vicinity of the lesion center 60, the hardness of the tissue is gradually or stepwise increasing towards the center of the lesion. The detachable extension comprises a heart-shaped tip 121, a flexible neck 122 and an elastic sleeve 123. When the needle 12 is inserted into the breast and passes through the normal tissue, the needle is advanced along a straight line. If the needle comes across a harder region of the tissue mass, it starts to bend the tip of the extension 121 in the direction of the steepest increase of the tissue hardness. The side-walls of the heart-shaped tip of the extension 121 experience different level of resistance depending on the local hardness of the tissue. Consequently, the resulting torque bends slightly the flexible neck 122 and the needle is directed closer to the harder area.

Although the invention herein has been described with respect to particular embodiments, it is understood that these embodiments are merely illustrative of the principles and applications of the present invention. For example, despite the fact that all of the above mentioned devices have been described as being combined with tactile imaging probes, it is also envisioned to have them produced as a stand-alone adapter, an add-on or snap-on device to the existing tactile imaging probes. Critically, the distance between the center point of the biopsy needle cannula has to be in fixed relationship to the pressure sensing surface of the tactile imaging probe. In that case, the computing means of the device can accurately calculate the correct deviation angle and distance and inform the operator of the need to correct thereof.

Moreover, in cases where it is advantageous to position the tactile imaging probe on one side of the soft tissue but advance the biopsy needle from another side, it is envisioned to have available a multiple number of attachment holders for the biopsy gun ball and socket joint. Such holders are made with known distance and spatial orientation between the pressure sensing surface of the imaging probe and the ball and socket joint of the needle guiding cannula.

It is therefore to be understood that numerous modifications may be made to the illustrative embodiments and that other arrangements may be devised without departing from the spirit and scope of the present invention as defined by the appended claims.

What is claimed is:

1. A device for guiding a needle of a soft tissue biopsy gun using a tactile imaging probe equipped with a pressure sensing surface, said device comprising:
a guiding cannula adapted to accept said biopsy needle for a sliding reciprocal motion therein,
a joint means supporting said guiding cannula at a center point in a fixed spatial relationship to said pressure sensing surface of said probe, said joint means adapted to allow said guiding cannula to rotate about said center point, said joint means equipped with a linear displacement sensor and an angular displacement sensor,
a computing means for accepting real-time 2-D digital imaging data of said soft tissue from said pressure sensing surface and simultaneously accepting a linear displacement and angular displacement data of said biopsy needle within said guiding cannula from respectively said linear displacement and angular displacement sensors, said computing means further adapted to determine in real time a spatial location of a desired target site and a distance and angular deviation of a distal end of said biopsy needle from said target site, and
an indicator means to continuously indicate said distance and said angular deviation as calculated by said computing means.

2. A device for guiding a needle of a soft tissue biopsy gun, said needle having a distal end, said device comprising:
a tactile imaging probe equipped with a pressure sensing surface,
a guiding cannula adapted to accept said needle for a sliding reciprocal motion therein,
a joint means supporting said guiding cannula at a center point in a fixed spatial relationship to said pressure sensing surface, said joint means adapted to allow said guiding cannula to rotate about said center point, said joint means equipped with a linear displacement sensor and an angular displacement sensor,
a computing means for accepting real-time 2-D digital imaging data of said soft tissue from said pressure sensing surface and simultaneously accepting a linear displacement and angular displacement data of said biopsy needle within said guiding cannula from respectively said linear displacement and angular displacement sensors, said computing means further adapted to determine in real time a spatial location of a desired target site and a distance and angular deviation of said distal end of said needle from said target site, and
an indicator means to continuously indicate said distance and said angular deviation as calculated by said computing means.

3. The device as in claim 2, wherein said joint means is a ball and socket joint.

4. The device as in claim 2, wherein indicator means producing an audio signal.

5. The device as in claim 4, wherein said indicator means adapted to change a frequency of said audio signal to indicate said angular deviation and a loudness of said audio signal to indicate said distance.

6. The device as in claim 2, wherein said indicator means is a visual display illustrating the position of said target site and said distal end of said biopsy needle respectively.

7. The device as in claim 6, wherein said visual display is adapted to display said distal end of said needle and said target site positions in two-dimensional projections.

8. The device as in claim 6, wherein said visual display is adapted to display said distal end of said needle and said target site positions in two orthogonal planes, said distance between said distal end of said needle and said target site is indicated by a circle having a diameter corresponding to said distance.

9. The device as in claim 8, wherein said two orthogonal planes are oriented respectively parallel and perpendicular to a skin covering said soft tissue.

10. The device as in claim 6, wherein said visual display is adapted to display said distal end of said needle and said target site in a three-dimensional projection.

11. The device as in claim 2, wherein said indicator means producing a light signal.

12. The device as in claim 11, wherein said indicator means adapted to change two independent parameters of said light to indicate said distance and angular deviation, said two parameters selected from a group of parameters consisting of color, brightness and pulse repetition rate.

13. The device as in claim 2, wherein said device further equipped with a data transmission cable, said computing means and said indicator means are supported by a personal computer.

14. The device as in claim 2, wherein said pressure sensing surface is covered with a disposable membrane.

15. The device as in claim 2, wherein said center point is located at said pressure sensing surface of said probe, said device further equipped with a mechanical advancement means for sliding said needle down into said soft tissue, said mechanical advancement means equipped with a linear displacement sensor.

16. The device as in claim 15, wherein said mechanical advancement means are a gear mechanism activated by a thumb-wheel.

17. The device as in claim 2, wherein said needle further equipped with a self-guiding extension at its distal end, said extension including a flexible neck terminating with a heart-shaped tip.

18. A method for guiding a needle of a soft tissue biopsy gun comprising the steps of:
a. providing a tactile imaging probe equipped with a pressure sensing surface,
b. supporting said needle at a center point located in a fixed spatial relationship to said pressure sensing surface,
c. pressing said tactile imaging probe against said soft tissue and moving its pressure sensing surface about thereof to acquire a series of 2-D pressure sensing images,
d. determining a location of a target site within said soft tissue relative to said pressure sensing surface,
e. determining of location of a distal end of said needle and its angular direction relative to location of said pressure sensing surface,
f. calculating the angular deviation between the direction of said needle and the direction from said center point to said target site,
g. calculating the distance between said distal end of said needle and said target site relative to said center point,
h. continuously indicating in real time of said angular deviation and said distance to assist in guiding of said needle towards said target site.

19. The method as in claim 20 further including activating said biopsy gun once the distance and the angular deviation are indicated to be equal or less than the acceptable predetermined values confirming therefore that the distal end of said needle is located at the target site.

20. The method as in claim 20, wherein step “d” further includes detecting a lesion and calculating its spatial position relative to said pressure sensing surface by analyzing spatial and temporal variations of said 2-D pressure sensing images.
